(19) United States
(12) Patent Application Publication
(10) Pub. No.: US 2005/0084505 A1
(43) Pub. Date: Apr. 21, 2005

(54) Utilization of ubiquinones for influencing the effect of histamine

(76) Inventors: Sylvana P Muller, Halle (DE); Jurgen Koch, Wiesbaden (DE)

Correspondence Address: JACOBSON HOLMAN PLLC, 400 SEVENTH STREET N.W., SUITE 600, WASHINGTON, DC, 20004, US

(21) Appl. No.: 10/506,423

(22) PCT Filed: Mar. 10, 2003
(86) PCT No.: PCT/EP03/02427

(30) Foreign Application Priority Data
Mar. 9, 2002 (DE) 102 10 447.6

(51) Int. Cl. A61K 31/12; A61K 9/00

(52) U.S. Cl. 424/400; 514/690

(57) ABSTRACT

Use of ubiquinones for reducing histamine activity.

The present invention relates to the use of ubiquinones for treating inflammatory diseases and influencing the activity of histamines.

Ubiquinones are prenylated quinones which are wide-spread in the animal and plant kingdoms. They are derivatives of 2,3-dimethoxy-5-methyl-1,4-benzo-quinone which comprise isoprene units linearly interlinked in six position. Depending on the number of isoprene units, the ubiquinones are designated as Q-1, Q-2, Q-3 etc. In most mammals including humans, Q-10 (2,3-dimethoxy-5-methyl-6-decaprenyl-1,4-benzoquinone) is prevailing. Ubiquinones serve as electron carriers in the respiratory chain, and they participate in the cyclic oxidation and reduction of substrates in the citric acid cycle. Ubiquinones Qₙ are a precondition for the energy supply of all cells. The oxidative stress which arises, inter alia, from a high oxygen consumption causes damage to the membranes of mitochondria and cells, which results in acute or degenerative disorders.

To date, ubiquinones have been used in the therapy of cardiac diseases.

WO 00/47192 discloses the use of ubiquinones for the treatment of pain.

Histamines are among the central mediators of inflammatory reactions. Usually, for example, in the activation of mast cells by immunological stimuli, preformed mediators such as histamine are released from granula by exocytosis, and simultaneously prostaglandine, SRS-A and other leukotrienes are newly synthesized. In Arzneimittelforschung 35 (1), No. 6 (1985), page 929, Ishihara et al. describe the effect of ubiquinone Q-10 on the release of histamines or SRS-A (slow-releasing substance of anaphylaxis) from prepared lung tissue and trachea pieces of guinea pigs. The histamine release upon the action of γ-globulin on lung tissue has been examined, and it was found that the secretion of histamines and SRS-A was reduced. The contraction of lung tissue by added histamine was increased when coenzymes Q10 were continuously administered. The tissue was not aerated.

In contrast, it has now been found that ubiquinones can reduce the activity of histamine and other mediators.

Ubiquinones within the meaning of this application comprise ubiquinone Qₙ compounds wherein n preferably ranges from 8 to 10. Ubiquinones further comprise ubihydroquinones, which are in an equilibrium with the ubiquinones, as well as simple esters of ubihydroquinones with short-chained carboxylic acids having from 8 to 10 carbon atoms, for example, acetic acid, propionic acid or butyric acid, which are converted to the corresponding ubiquinones upon administration. In a preferred embodiment, the ubiquinone is 2,3-dimethoxy-5-methyl-6-decaprenyl-1,4-benzoquinone.

Agents for inhibiting histamine release are known. Surprisingly, however, it has now been found that ubiquinones are capable of reducing the activity of histamines. The extent of this reduction can be such as to result in an elimination of the activity of histamine. In contrast to other agents, histamine is actually released, but its activity is reduced. Such agents have not been described to date. Thus, possible applications include all conditions where an activity of histamine is undesirable, irrespective of the cause of histamine secretion.

Therefore, the invention relates, on the one hand, to the use of ubiquinone for preparing a medicament for the treatment of diseases. Particularly suitable diseases are bronchopulmonary inflammatory diseases since bronchoconstriction can be reduced by administering ubiquinones. Therefore, ubiquinone is suitable for the treatment of bronchitis, pneumonia, hay fever, allergic asthma and skin diseases. The activity of histamine in allergic asthma causes rigidity and stiffness of the lung tissue. By inhibiting the activity of histamine, ubiquinones are a suitable therapeutic agent.

Also in cases of multiple chemical sensitivity (MCS), ubiquinones are suitable for treatment.

In a further aspect of the invention, ubiquinone can be used for the preparation of a medicament for reducing the activity of histamine after histamine release. Histamine release is a central step in many body reactions, for example, in hypersensitivity reactions of the immediate type (type 1), in endotoxin shock, in burns, in renal insufficiency and in histamine release induced by pharmaceuticals.

Such pharmaceutical-induced histamine releases can be initiated by various agents. They are frequent in connection with the use of D-tubocurarin, suxa-methonium, opiates, X-ray contrast agents, narcotics and chloroquine.

The ubiquinones can be employed in forms adapted to the respective instance of application. Depending on the kind of application, oral, inhalatory, intravenous, intraarterial, subcutaneous, intracutaneous, intramuscular or topical application forms can be chosen. Particularly suitable are application forms in which ubiquinone is in the form of nanoparticles having a size of from 10 to 1000 nm.

The invention further relates to the use of ubiquinone for preparing food supplements and cosmetic preparations for reducing histamine activity.

The invention further relates to the use of ubiquinone for influencing the activity of histamine and other mediators. Other mediators are those mediators which are also released as a response in inflammatory processes, especially PAF (platelet activating factor), leukotrienes as well as SRS-A (slow-reacting substance of anaphylaxis).

Thus, ubiquinones are suitable for influencing bronchoconstriction of the lung, the pulmonary vascular resistance, elasticity of the lung, bowel contraction, epithelial contractions, vascular contractions, for reducing catecholamine release from the adrenal medulla, histamine-caused itching or pain at nerve ends, excessive secretion of gastric juice, tachycardia and the positive-ionotropic effect on the heart.

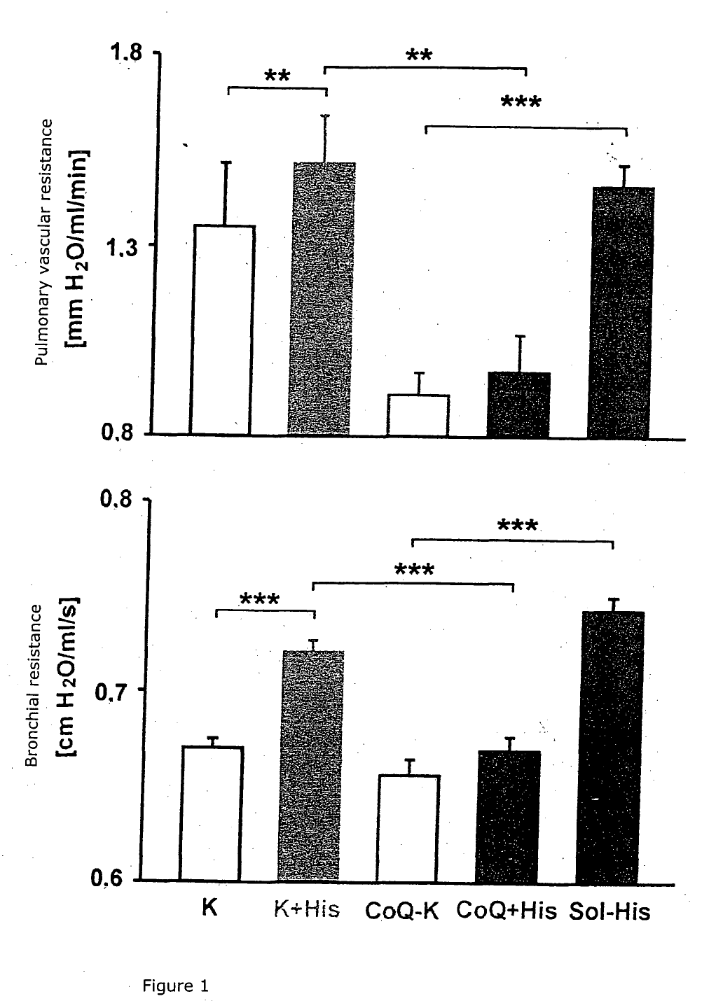

FIG. 1 shows the effect of CoQ-10 in the experiment according to Example 1.

K=control

His=histamine

CoQ=coenzyme Q

Sol=solvent (ethanol, glycerol, lecithin, Tris buffer pH 7.4).

***=p<0.001

**=p<0.01

The mode of action of ubiquinone is further illustrated by the following Example.

### EXAMPLE 1

Isolated, perfused and ventilated lungs from adult rats (280 to 330 g body weight) were injected with 50 pg of histamine per lung. After 2 to 3 hours, this caused constriction of the bronchial and pulmonary vessels with reduction of the lung extensibility. The previous addition of 2.7 ppm of CoQ-10 in the perfusate completely prevented the delayed histamine reaction and decreased the spontaneous vasoconstriction of the lung vessels even below the control perfusion.

1. Use of ubiquinones for preparing a medicament for reducing histamine activity in diseases selected from bronchitis, pneumonia, allergic asthma, multiple chemical sensitivity, hay fever, hypersensitivity reactions of the immediate type (type 1), endotoxin shock, histamine release induced by pharmaceuticals, skin diseases, renal insufficiency and burns.

2. The use according to claim 1, characterized in that said pharmaceutical-induced histamine release is caused by D-tubocurarin, suxamethonium, opiates, X-ray contrast agents, narcotics and/or chloroquine.

3. The use according to claim 1, characterized in that ubiquinone is in the form of nanoparticles having a size of from 10 to 1000 nm.

4. The use according to claim 1, characterized in that said medicament is in a form adapted for oral, inhalatory, intravenous, intraarterial, subcutaneous, intracutaneous, intramuscular or topical application.

5. Use of ubiquinones for influencing the activity of histamines and other mediators.

6. The use according to claim 5, characterized in that said mediators are selected from the group consisting of PAF and leukotrienes.

7. The use according to claim 5, characterized in that ubiquinones are used for influencing bronchoconstriction of the lung, pulmonary vascular resistance, elasticity of the lung, bowel contractions, epithelial constriction, vascular contractions, for reducing catecholamine release from the adrenal medulla and/or histamine-caused itching or pain at nerve ends, excessive secretion of gastric juice, tachycardia and/or the positive-ionotropic effect on the heart.

8. The use according to claim 5, characterized in that said ubiquinone is in the form of nanoparticles having a size of from 10 to 1000 nm.

9. The use according to claim 1, characterized in that said ubiquinone is 2,3-dimethoxy-5-methyl-6-decaprenyl-1,4-benzoquinone.

10. Use of ubiquinones for preparing a food supplement or cosmetic preparation for reducing histamine activity.
